(19) United States
(12) Patent Application Publication
(10) Pub. No.: US 2021/0170393 A1
(43) Pub. Date: Jun. 10, 2021

(54) SAMPLE TEST CARD AND SAMPLE LOADING METHOD THEREOF

(71) Applicant: Hefei Hengxing Technology Development Co., Ltd., Anhui (CN)

(72) Inventors: Ying Zhou, Anhui (CN); Yongqiang Wang, Anhui (CN); Haokun Zhou, Anhui (CN)

(21) Appl. No.: 17/111,604

(22) Filed: Dec. 4, 2020

(30) Foreign Application Priority Data
Dec. 6, 2019 (CN) 201911242130.5

(51) Int. Cl. B01L 3/00; G01N 21/01; C12M 1/12; C12M 1/00; B01L 1/02; C12M 1/107

(52) U.S. Cl. CPC B01L 3/502; B01L 2200/0689; C12M 23/04; C12M 23/22; C12M 23/34; B01L 1/025; C12M 23/36; B01L 3/5085; B01L 2300/0816; B01L 2300/0829; B01L 2400/086; B01L 2300/168; B01L 2400/049; B01L 2200/141; G01N 21/01

(57) ABSTRACT

The present disclosure provides a sample test card and a sample loading method thereof, relates to the technical field of microbiological testing. The present disclosure includes sample wells arranged in an array; the sample wells are connected together through a flow channel network, and the sample wells are filled with a sample through a unified intake port. Sample filling is completed by vacuuming; during filling, a liquid sample is firstly filled, and air or other inert gas or insoluble liquid is filled; liquid sample volume and air volume are formed in proportion in the sample wells. In the present disclosure, the filled liquid sample is controlled not to fill the sample wells completely, and there are sufficient air space in sample wells, so that the sample wells are independent from each other to avoid contamination, and more sample wells can be arranged on the test card with the same size.

### CROSS REFERENCE TO RELATED APPLICATION

This application claims benefit of priority from Chinese Patent Application No. 201911242130.5, filed Dec. 6, 2019. The contents of this application are incorporated herein by reference.

### TECHNICAL FIELD

The present disclosure belongs to the technical field of microbiological testing, and in particular relates to a sample test card and a sample loading method thereof.

### BACKGROUND

The sample test card has been used by the bioMérieux Corporate in automatic instrument that use the principle of optical intensity detection, such as a test card described in U.S. Pat. No. 5,609,828, incorporated by reference herein; in addition, there is a Chinese patent titled “IMPROVED SAMPLE TEST CARD,” with Publication No. CN103154744B, incorporated by reference herein and a U.S. Pat. No. 10,252,262B2, incorporated by reference herein.

U.S. Pat. No. 5,609,828 reduces the possibility of inter-well contamination by increasing the well-to-well distance between flow paths; the latest patents CN103154744B and U.S. Pat. No. 10,252,262B2 propose a new and improved method for the previous patent, introducing flow and overflow reservoirs connected to a fluid channel network. The overflow reservoir can absorb the fluid sample in the flow path and the flow reservoir, so that the flow reservoir and the flow path are filled with air, thereby acting as an air barrier or airlock to prevent well-to-well contamination; because the flow path between the wells is substantially shortened, more sample wells can be arranged on a test card of the same size to meet the needs of the total number of sample wells. Integrated with the prior art and patents, the key feature is: the sample wells must be filled with sample and the inter-well contamination is reduced by increasing the well-to-well flow path distance; alternatively, the inter-well contamination is prevented by filling air with the overflow reservoir through the air barrier or airlock; the sample wells are sealed with oxygen-permeable transparent tapes to ensure the growth of microorganisms in the sample wells filled with fluid sample.

Based on the current situation, the existing products and disclosures have the following deficiencies: Test cards adopt transmission detection, and patents require that the sample well must be filled completely during sample loading to prevent the instability of detection caused by the presence of bubbles. In order to grow microorganisms in the sample wells, the transparent sealing tape must be air-permeable, thereby increasing the process difficulty of practical products. In addition, when the sample wells are full, the metabolic gases generated by the growth of microorganisms in the cells leads to changes of pressure in the wells; the liquid sample may be pressed back into the very small flow reservoirs, so that the liquid between the wells is reconnected together, resulting in faster inter-well contamination. The existing patents No. CN103154744B and U.S. Pat. No. 10,252,262B2 have defects and has not been applied in products so far; further, sample loading by vacuum filling cannot meet the requirement of filling the sample wells completely in plateau regions, so the existing products by bioMérieux Corporate clearly limit the applicable altitude; in addition, prolonged microbiological testing time has also been a challenge that restricts the promotion and application. Clinically, there is an urgent need to improve testing speed.

The present disclosure proposes a novel design idea: The sample wells are not filled with liquid samples, and the air in each sample well isolates each sample well to make each sample well completely independent and completely solve the inter-well contamination; in addition, the air present in the sample well provides the oxygen needed for the growth of microorganisms, and a sealing film does not need to have air permeability; furthermore, liquid sample loading is not restricted by the ambient atmospheric pressure, and products can be applied to regions of all altitudes. The present disclosure is particularly provided with a thin layer microscopic observation chamber for microscopic imaging, realizing rapid testing, and introducing a new testing method for this field.

Compared with the prior art, the present disclosure has the advantages of simple process, reliable performance, strong practicability, high integration, low cost, etc. Especially through the use of microscopic observation and image processing technology, clinically rapid drug susceptibility analysis can be achieved; the methodological change from the existing macroscopic turbidimetry to microscopic morphological analysis has a qualitative breakthrough in improving the testing speed. The present disclosure has novelty in the fields of microbial sample loading and testing.

### SUMMARY

An aspect of the present disclosure is to provide a sample test card and a sample loading method thereof. Sample wells are filled with a liquid sample through a uniform sample intake port during sample loading. Sample filling is completed by vacuuming; the liquid sample is firstly filled, and air or other inert gas or non-water-soluble liquid is filled; liquid sample volume and air volume are formed in proportion in the sample wells. In the present disclosure, the state after the completion of the sample loading is that: the liquid sample does not fill the sample wells completely, and there are sufficient air space in sample wells, so that the sample wells are independent from each other to avoid contamination, and desired air volume is provided for the growth of a biological sample in the sample wells in a closed state.

To solve the above technical problems, the present disclosure is achieved by the following technical solutions:

The present disclosure provides a sample test card, where the sample test card has a slab structure, a plurality of sample wells, a fluid intake port and a fluid flow channel network are sealed and arranged inside the sample test card, and the fluid flow channel network communicates with the fluid intake port and the sample wells; transparent blocks are arranged inside the sample wells of the sample test card; the transparent blocks divide the sample wells into storage chambers and observation chambers.

Further, both the sample test card and the transparent blocks may be made of transparent materials or transparent film materials.

Further, the sample well may be composed of a storage chamber and an observation chamber; the observation chamber may be a thin layer structure, and the thin layer of the observation chamber may be transparent in the vertical direction and may be used for microscopic observation.

A sample loading method of a sample test card is provided, where a liquid test sample is incompletely filled in the sample well, gas is present in the upper part of the sample well and the flow channel network, to achieve the proportional relationship between the amount of liquid test sample in the sample well and the gas volume; including the following steps:

SS01, providing a liquid test sample and a sample test card;

SS02, inserting one end of a fine pipette into a fluid intake port on the sample test card, and connecting the other end of the fine pipette to the liquid test sample in a test tube; sealing the sample test card, liquid test sample, test tube, and a bracket for placement in a sample loading chamber for vacuuming, so that air in each sample well, flow channel network and fine pipette in the sample test card is discharged through the liquid test sample, and a vacuum chamber reaches a certain vacuum degree;

SS03, after vacuuming to a certain vacuum degree, slowly introducing air into the vacuum chamber so that the liquid test sample in the test tube is sucked into the flow channel through the fine pipette to reach the sample well, and filling the sample well with the liquid test sample; in the process of introducing air, achieving the desired requirement of the vacuum degree to complete partial filling of the sample well with the liquid test sample, and separating the liquid test sample in the fine pipette from that in the test tube; and

SS04, continuing to introduce air into the vacuum chamber until the pressure in the vacuum chamber is consistent with the atmospheric pressure; where at this time, the liquid test sample remaining in the flow channel network flows into the sample well, and air flows into the network and the sample well.

Further, the vacuum chamber in SS03 may undergo deflation, and the vacuum degree in the deflation of the vacuum chamber may be controlled so that the liquid test sample slowly flows into the sample well, and the volume of the sample filled in the sample well may reach a proportion required in the entire sample well.

Further, the fine pipette may be inserted into a water-insoluble solvent with smaller specific gravity than water in another test tube after separating the liquid test sample in the fine pipette from that in the test tube in SS03. In SS04, the solvent may be finally left in the flow channel network and the upper part of the sample well.

Further, an inert gas or a gas mixture without oxygen may be introduced into the vacuum chamber in both SS03 and SS04 when used in an anaerobic microbial test.

Further, a total volume of the liquid test sample provided may be less than that of all sample wells; according to the above sample loading method by vacuuming, first the liquid test sample may be filled into the test card, and next, when the liquid test sample is used up, the test card may be filled with air until the end of the sample loading. The flow channel network and the upper part of the sample well may be filled with air, so that the volume of the sample filled in the sample well reaches a proportion required in the entire sample well.

The present disclosure is a sample test card. The sample test card is substantially rectangular in shape. The sample wells are arranged in horizontal rows and vertical columns. A main flow path and branch flow paths constitute a flow channel network, which connects each sample well together and is connected to the fluid intake port; biological samples may be direct samples from patients or sample solutions processed in other manners; the sample test card is used in a horizontal position.

Herein, the sample test card may include: flowing a liquid test sample from the intake port through the main flow path and the branch flow into each sample well.

Herein, the test card may be preferably assembled to generate a card body and cover plates into a complete card to ensure that the sample wells and the flow channel network are in a sealed state. In addition, to ensure the reliability of the sealing, an annular sealing groove may be arranged around the sample well group.

Herein, the card body may be preferably arrayed with cylindrical cavities, that is, sample wells; a main flow path groove and branch flow path grooves may be distributed together on one surface to form a flow path network, which is connected to each sample well cavity and a cone well on the other side; in addition, an annular rib may be provided.

Herein, the cover plates may be preferably arrayed with cylindrical posts; a rib-like network may be distributed on one plane, and in addition, an annular groove may be provided.

Herein, there may be a corresponding relationship between the card body and the cover plates in structure and shape: the sample well grooves on the card body may correspond to the ribs on the cover plates, the groove network may correspond to the rib network, and the annular ribs may correspond to the annular grooves. The card body and the cover plate may be pressed into the test card according to the corresponding positions. The edge of the sample well orifice and the flow channel may be fitted tightly by pressing the rib and the groove to form a sealed sample well, a sealed flow channel and a sealed annular ring.

The present disclosure adopts a pressing method to complete the assembly of the test card, with a simple processing method. In order to further ensure the overall sealing performance of the sample well and the flow channel, a method for pressing after bonding an O-ring may be used.

The present disclosure has the following beneficial effects:

In the present disclosure, sample wells are filled with a liquid sample through a uniform sample intake port during sample loading. Sample filling is completed by vacuuming; the liquid sample is firstly filled, and air or other inert gas or non-water-soluble liquid is filled; liquid sample volume and air volume are formed in proportion in the sample wells. In the present disclosure, the state after the completion of the sample loading is that: the liquid sample does not fill the sample wells completely, and there are sufficient air space in sample wells, so that the sample wells are independent from each other to avoid contamination, and desired air is provided for the growth of a biological sample in the sample wells in a closed state.

Of course, any product implementing the present disclosure does not necessarily need to achieve all the preceding advantages at the same time.

### BRIEF DESCRIPTION OF THE DRAWINGS

In order to more clearly explain the technical solutions of the embodiments of the present disclosure, the drawings needed to describe the embodiments will be briefly introduced below. Obviously, the drawings in the following description are only some embodiments of the present disclosure, and other drawings can be obtained by those of ordinary skill in the art based on these drawings without creative work.

FIG. 1 is a top view of the structure of a sample test card in Embodiment 1 of the present disclosure;

FIG. 2 is an exploded schematic cross-sectional view of a sample test card in Embodiment 1 of the present disclosure;

FIG. 3 is a schematic cross-sectional view of a sample test card in Embodiment 1 of the present disclosure;

FIG. 4 is a schematic cross-sectional view of a sample well in Embodiment 1 of the present disclosure;

FIG. 5 is an exploded structural diagram of a sample test card in Embodiment 2 of the present disclosure;

FIG. 6 is a top view of the structure of a sample test card in Embodiment 2 of the present disclosure;

FIG. 7 is a schematic cross-sectional view of a sample test card in Embodiment 2 of the present disclosure;

FIG. 8 is a schematic cross-sectional view of a sample well in Example 2 of the present disclosure;

In the drawings, a list of parts represented by each reference number is as follows:

1—Sample test card, 2—sample well, 3—fluid intake port, 4—fluid flow channel network, 5—transparent block, 6—storage chamber, and 7—observation chamber.

### DETAILED DESCRIPTION

The technical solutions in the embodiments of the present disclosure will be described clearly and completely in conjunction with the drawings in the embodiments of the present disclosure. Obviously, the described embodiments are only a part of, not all of, the embodiments. Based on the embodiments of the present disclosure, all other embodiments obtained by a person of ordinary skill in the art without creative work shall fall within the protection scope of the present disclosure

### Embodiment 1

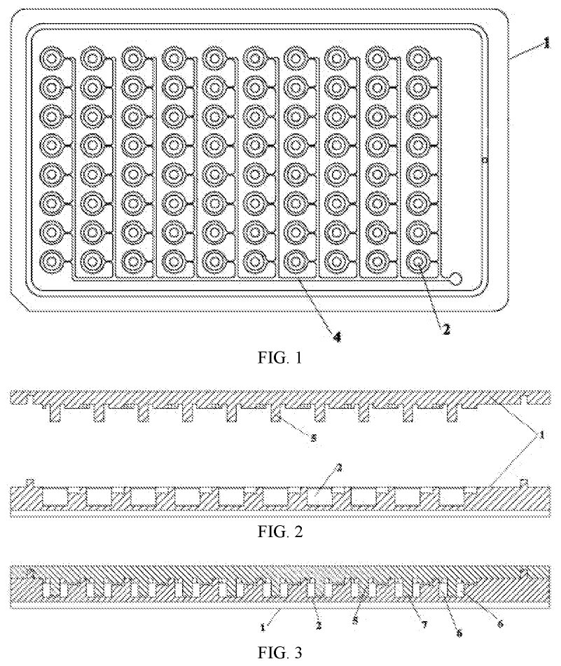

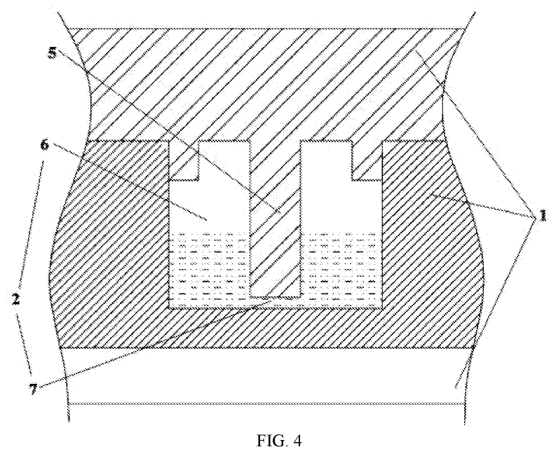

Please refer to FIGS. 1 to 4. The present disclosure provides a sample test card. The sample test card 1 has a slab structure, a plurality of sample wells 2, a fluid intake port 3 and a fluid flow channel network 4 are sealed and arranged inside the sample test card 1, and the fluid flow channel network 4 communicates with the fluid intake port 3 and the sample wells 2; transparent blocks 5 are arranged inside the sample wells 2 of the sample test card 1; the transparent blocks 5 divide the sample wells 2 into storage chambers 6 and observation chambers 7.

The storage chambers 6 are communicated with the observation chambers 7, the observation chambers 7 are in the center of the sample wells, the observation chambers 7 are 0.1-0.5 mm thin layers, so as to ensure a better microscopic imaging effect; The transparent blocks 5 of the upper cover plate in the sample test card 1 have a light-guide effect; when using microscopic observation, the structure in the vertical direction of the observation layer should be ensured to have better transparency.

Preferably, both the sample test card 1 and the transparent blocks 5 may be made of transparent materials or transparent film materials.

Preferably, the sample well 2 may be composed of a storage chamber 6 and an observation chamber 7; the observation chamber 7 may be a thin layer structure, and the thin layer of the observation chamber may be transparent in the vertical direction and may be used for microscopic observation.

A sample loading method of a sample test card is provided, where a liquid test sample is incompletely filled in the sample well, gas is present in the upper part of the sample well and the flow channel network, to achieve the proportional relationship between the amount of liquid test sample in the sample well and the gas volume; including the following steps:

SS01, providing a liquid test sample and a sample test card;

SS02, inserting one end of a fine pipette into a fluid intake port on the sample test card, and connecting the other end of the fine pipette to the liquid test sample in a test tube; sealing the sample test card, liquid test sample, test tube, and a bracket for placement in a sample loading chamber for vacuuming, so that air in each sample well, flow channel network and fine pipette in the sample test card is discharged through the liquid test sample, and a vacuum chamber reaches a certain vacuum degree;

SS03, after vacuuming to a certain vacuum degree, slowly introducing air into the vacuum chamber so that the liquid test sample in the test tube is sucked into the flow channel through the fine pipette to reach the sample well, and filling the sample well with the liquid test sample; in the process of introducing air, achieving the desired requirement of the vacuum degree to complete partial filling of the sample well with the liquid test sample, and separating the liquid test sample in the fine pipette from that in the test tube; and

SS04, continuing to introduce air into the vacuum chamber until the pressure in the vacuum chamber is consistent with the atmospheric pressure; where at this time, the liquid test sample remaining in the flow channel network flows into the sample well, and air flows into the network and the sample well.

Preferably, the vacuum chamber in SS03 may undergo deflation, and the vacuum degree in the deflation of the vacuum chamber may be controlled so that the liquid test sample slowly flows into the sample well, and the volume of the sample filled in the sample well may reach a proportion required in the entire sample well.

Preferably, the fine pipette may be inserted into a water-insoluble solvent with smaller specific gravity than water in another test tube after separating the liquid test sample in the fine pipette from that in the test tube in SS03. In SS04, the solvent may be finally left in the flow channel network and the upper part of the sample well.

Preferably, an inert gas or a gas mixture without oxygen may be introduced into the vacuum chamber in both SS03 and SS04 when used in an anaerobic microbial test.

Preferably, a total volume of the liquid test sample provided may be less than that of all sample wells; according to the above sample loading method by vacuuming, first the liquid test sample may be filled into the test card, and next, when the liquid test sample is used up, the test card may be filled with air until the end of the sample loading. The flow channel network and the upper part of the sample well may be filled with air, so that the volume of the sample filled in the sample well reaches a proportion required in the entire sample well.

Sample loading of the test card of the present disclosure: The sample of the test card is liquid, the intake port of the test card is inserted into a fine pipette, the other end of the pipette is placed in a test tube or container containing a liquid sample, and the test card is placed flat; the three are in a vacuum chamber, which is vacuumed to a pressure of 0.7-0.9 PSIA; the vacuum chamber and the sample wells and flow channels inside the test card are under vacuum negative pressure, and air is introduced into the vacuum chamber; at this time, the liquid sample is sucked into pipette from the port inserted into the test tube, introduces through the intake port, main flow path of the card, and branch flow paths, and finally reaches the sample wells. When the loading volume of the sample well reaches the desired amount, the intake port of the pipette is removed from the liquid sample in the test tube or the pipette is pulled up from the intake port of the test card, and air is introduced into the vacuum chamber continuously; at this time, air enters the intake port of the test card, the main flow path, and branch flow paths and finally reaches the sample wells. As air is introduced slowly and continuously, air is continuously filled into the sample wells until the vacuum chamber is released to atmospheric pressure, and the entire sample loading process ends. The control of the amount of sample loaded into the sample well is achieved by detecting the pressure in the vacuum chamber; in addition, and control of the speed of introducing air ensures the consistency of the amount of sample loaded in each sample well.

Herein, air filled in the latter part of the sample loading process makes the main flow path, branch flow paths, and the upper half of the sample wells be filled with air, so as to completely isolate each sample well. This method of not fill the sample wells with liquid sample completely has a better isolation effect, which is more reliable and more convenient to avoid inter-well contamination. In addition, the flow channel may be short enough, and the sample wells are arranged more compactly. Compared with the test card of the same size in the prior art, more sample wells may be arranged to meet the testing requirements.

Herein, in case of an anaerobic biological sample, the gas released after vacuuming after sample loading may be an inert gas or a gas mixture without oxygen to ensure the growth of microorganisms in an oxygen-free state. When the test card is preferably used for antibiotic drug susceptibility test, a powder containing antibiotics is attached to the sample well, and is controllably located at the bottom of the sample well, which improves the hydrophilicity of the bottom; the liquid sample first reliably reaches and fills the bottom when the test card is loaded, so that there is no air bubble in the observation chamber.

Herein, when another method for controlling the sample volume of sample well of the test card is used: according to the proportional relationship between the liquid sample volume and the air volume in the sample well required by the test card, the total amount of samples required by all sample wells is calculated, and the same amount of total liquid sample is accurately provided when loading the sample; the sample is loaded in the same way. When ensuring that one end of the fine pipette is inserted into the bottom of the liquid sample test tube, the liquid sample is first sucked when loading the sample; when all the liquid samples are sucked, the vacuum chamber still has a certain negative pressure, and air continues to be filled slowly until the vacuum chamber is released to atmospheric pressure. The entire sample loading process ends, so that the liquid sample volume and the gas volume in the sample well reach a predetermined proportional relationship to achieve an isolation effect between the sample wells.

Herein, when the test card of the present disclosure is used, after the sample is loaded, the intake port of the test card is closed to prevent biological contamination caused by the outflow of the sample.

Herein, the test card maintains a horizontal state during the sample loading process and the detection process in the instrument.

The test card of the present disclosure is especially used in the rapid drug susceptibility test of microscopic observation.

The production process of the test card of the present disclosure: The test card of the present disclosure is a carrier used to complete the detection of biological samples. Different powered reagents are attached to the sample wells. The attachment process of the powered reagents is the main production process of the test card. Firstly, the desired liquid reagent is added into the grooves of the card body, and the liquid reagent in the grooves of the card body is evaporated, lyophilized or dried by other means, so that the desired different reagents are attached to the sample wells. After drying, the card body and the cover plates are pressed together to complete the main production of the test card. When the test card is used, the liquid sample dissolves the powered reagents in the sample wells.

### Embodiment 2

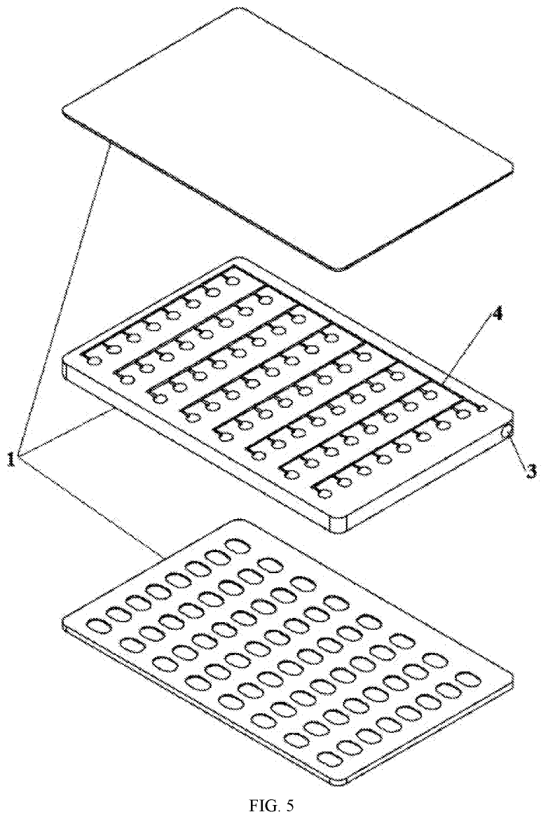

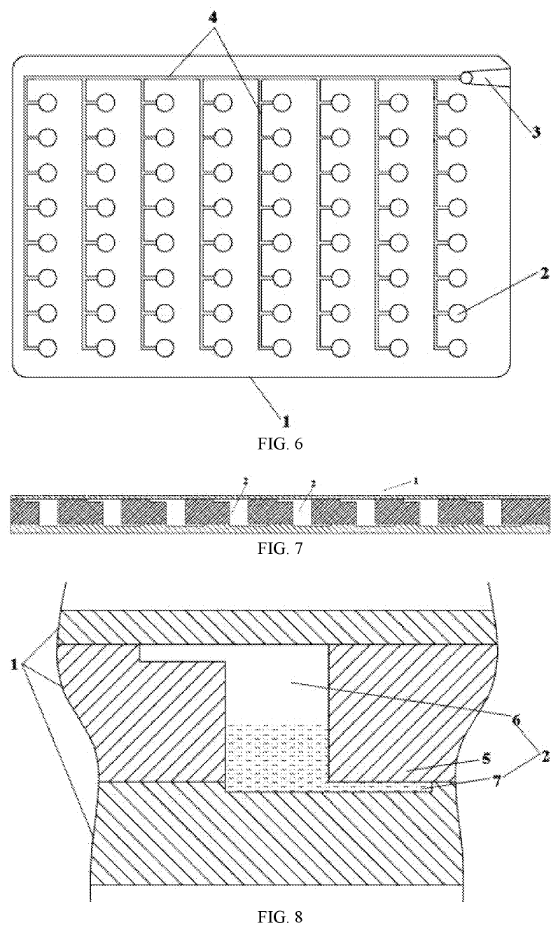

Please refer to FIGS. 5 to 8. The present disclosure provides a sample test card. The sample test card 1 has a slab structure, a plurality of sample wells 2, a fluid intake port 3 and a fluid flow channel network 4 are sealed and arranged inside the sample test card 1, and the fluid flow channel network 4 communicates with the fluid intake port 3 and the sample wells 2; transparent blocks 5 are arranged inside the sample wells 2 of the sample test card 1; the transparent blocks 5 divide the sample wells 2 into storage chambers 6 and observation chambers 7.

The storage chambers 6 are communicated with the observation chambers 7, the observation chambers 7 are on the sides of the sample wells, the observation chambers 7 are 0.1-0.5 mm thin layers, so as to ensure a better microscopic imaging effect; The transparent blocks 5 of the upper cover plate in the sample test card 1 have a light-guide effect; when using microscopic observation, the structure in the vertical direction of the observation layer should be ensured to have better transparency.

Preferably, both the sample test card 1 and the transparent blocks 5 may be made of transparent materials or transparent film materials.

Preferably, the sample well 2 may be composed of a storage chamber 6 and an observation chamber 7; the observation chamber 7 may be a thin layer structure, and the thin layer of the observation chamber may be transparent in the vertical direction and may be used for microscopic observation.

Herein, for the upper and lower surfaces defined by the card body 1, a plurality of sample wells 2 are distributed between the upper and lower surfaces, a main flow path and branch flow paths constitute a flow channel network arranged on the upper surface and connected to each sample well 2 and the fluid intake port 3.

Herein, a lower cover plate 2 is a slab; grooves are distributed on the upper surface, and the grooves correspond to the sample wells in the card body; the lower surface of the card body 1 is bonded to the upper surface of the lower cover plate, and the sample wells and the grooves on the lower surface form bottom-closed sample wells and thin layer observation chambers; the thickness of the observation chambers is formed by the gap of the corresponding parts, and preferably the thickness may be 0.1-0.5 mm, used for microscopic observation of microorganisms; to introduce an image processing method, especially to realize quick identification of the testing effect of antibiotics, all the components of the test card in the vertical direction of the thin layer of the observation chamber are transparent, which are used for microscopic observation of the light passing through the light path.

The assembly of the test card: The lower surface of the card body 1 is bonded to the upper surface of the lower cover plate, and the upper surface of the card body 1 is pasted with a transparent sealing film to form a sealed flow channel network and sealed sample wells.

The production process of the test card of the present disclosure: First, the card body 1 and the lower cover plate are pasted together to form sample wells opened above; desired reagents are added to the sample wells, evaporated, lyophilized or dried by other means to make powdered reagents be attached to the surface of the sample wells, and a sealing film is attached to the upper surface of the test card to complete the main production of the test card. When in use, the liquid sample dissolves the powdered reagents in the sample wells.

In the description of this specification, the descriptions referring to the terms “one embodiment”, “example”, “specific example”, etc. mean that the specific features, structures, materials, or characteristics described in conjunction with the embodiment or example are included in at least one embodiment or example of the present disclosure. In this specification, the schematic representation of the above terms does not necessarily refer to the same embodiment or example. Moreover, the specific features, structures, materials, or characteristics described may be combined in any one or more embodiments or examples in any suitable manner.

The preferred embodiments of the present disclosure disclosed above are only used to help illustrate the present disclosure. The preferred embodiments neither describe all the details in detail, nor limit the present disclosure to the specific embodiments described. Obviously, a plurality of modifications and changes can be made according to the content of this specification. This specification selects and specifically describes these embodiments, in order to better explain the principle and practical application of the present disclosure, so that those skilled in the art can well understand and use the present disclosure. The present disclosure is only limited by the claims, full scope thereof and equivalents.

1. A sample loading method of a sample test card, wherein the sample test card has a slab structure; a plurality of sample wells, a fluid intake port and a fluid flow channel network are sealed and arranged inside the sample test card, and the fluid flow channel network communicates with the fluid intake port and the sample wells; transparent blocks are arranged inside the sample wells of the sample test card;
the transparent blocks divide the sample wells into storage chambers and observation chambers;
both the sample test card and the transparent blocks are made of transparent materials
the sample well comprises a storage chamber and an observation chamber; the observation chamber is a thin layer structure, and the thin layer of the observation chamber is transparent in the vertical direction and is used for microscopic observation; and
a liquid test sample is incompletely filled in the sample well, gas is present in an upper part of the sample well and the flow channel network, to achieve a proportional relationship between an amount of liquid test sample in the sample well and a gas volume; and the method comprises the following steps:
SS01, providing a liquid test sample and a sample test card;
SS02, inserting one end of a fine pipette into a fluid intake port on the sample test card, and connecting the other end of the fine pipette to the liquid test sample in a test tube; sealing the sample test card, liquid test sample, test tube, and a bracket for placement in a sample loading chamber for vacuuming, so that air in each sample well, flow channel network and fine pipette in the sample test card is discharged through the liquid test sample, and a vacuum chamber reaches a certain vacuum degree;
SS03, after vacuuming to a certain vacuum degree, slowly introducing air into the vacuum chamber so that the liquid test sample in the test tube is sucked into the flow channel through the fine pipette to reach the sample well, and filling the sample well with the liquid test sample; in the process of introducing air, achieving the desired requirement of the vacuum degree to complete partial filling of the sample well with the liquid test sample, and separating the liquid test sample in the fine pipette from that in the test tube; and
SS04, continuing to introduce air into the vacuum chamber until the pressure in the vacuum chamber is consistent with the atmospheric pressure; wherein at this time, the liquid test sample remaining in the flow channel network flows into the sample well, and air flows into the flow channel network and the sample well.

2. (canceled)

3. (canceled)

4. (canceled)

5. The sample loading method of a sample test card according to claim 1, wherein the vacuum chamber in SS03 undergoes deflation, and the vacuum degree in the deflation of the vacuum chamber is controlled so that the liquid test sample slowly flows into the sample well, and the volume of the sample filled in the sample well reaches a proportion required in the entire sample well.

6. The sample loading method of a sample test card according to claim 1, wherein the fine pipette is further inserted into a water-insoluble solvent with smaller specific gravity than water in another test tube after separating the liquid test sample in the fine pipette from that in the test tube in SS03; in SS04, the solvent is finally left in the flow channel network and the upper part of the sample well.

7. The sample loading method of a sample test card according to claim 1, wherein, an inert gas or a gas mixture without oxygen is introduced into the vacuum chamber in both SS03 and SS04 when used in an anaerobic microbial test.

8. The sample loading method of a sample test card according to claim 1, wherein a total volume of the liquid test sample provided is less than that of all sample wells; first the liquid test sample is filled into the test card, and next, when the liquid test sample is used up, the test card is filled with air until the end of the sample loading; the flow channel network and the upper part of the sample well are filled with air, so that the volume of the sample filled in the sample well reaches a proportion required in the entire sample well.
